(19) United States
(12) Patent Application Publication
(10) Pub. No.: US 2014/0222148 A1
(43) Pub. Date: Aug. 7, 2014

(54) BRIDGE BUTTON FOR LIGAMENT RECONSTRUCTION

(71) Applicant: Padmaker Shinde, Nashik (IN)

(72) Inventor: Padmaker Shinde, Nashik (IN)

(21) Appl. No.: 14/240,614

(22) PCT Filed: Aug. 17, 2012
(86) PCT No.: PCT/IN2012/000555

§ 371 Date: Feb. 24, 2014

(30) Foreign Application Priority Data
Aug. 24, 2011 (IN) 2376/MUM/2011

(51) Int. Cl. A61F 2/08

(52) U.S. Cl. CPC A61F 2/0811; A61F 2002/0858
USPC 623/13.14

(57) ABSTRACT

A bridge button, for ligament reconstruction, said button being formed by an arcuate body of pre-defined dimensions such that said button being adapted to sit across a cavity in a bone and further adapted to operate between an operative open position and an operative closed position, said button comprises: a first shaft, and a co-axial second shaft co-operating with said first shaft in a manner such that said second shaft is adapted to be angularly displaced about said first shaft at their substantially co-axially located pivoting point, said second shaft being located operatively atop said first shaft.

### FIELD OF THE INVENTION

This invention relates to the field of biomedical engineering.

Particularly, this invention relates to the field of biomedical engineering related to ligament reconstruction.

Still particularly, this invention relates to mechanical fixtures for ligament reconstruction.

More particularly, this invention relates to a bridge button for ligament reconstruction.

### BACKGROUND OF THE INVENTION

Knee, in humans, support the entire body weight. It is hence susceptible to injury, apart from wear and tear. The knee is the largest joint in the human body. The knee joint joins the thigh with the leg and consists of two articulations: one between the femur and tibia, and one between the femur and patella. It provides flexion and extension movement apart from slight medial and lateral rotation.

The components of the knee include ligaments; which offer stability by limiting movements. Cruciate ligaments are ligaments which cross each other like the letter ‘X’. Although, they allow a large range of motion, they stabilize the knee. The cruciate ligaments of the knee are the anterior cruciate ligament (ACL) and the posterior cruciate ligament (PCL). The ACL is lateral and the PCL is medial.

The ACL originates from deep within the notch of the distal femur. Its proximal fibers fan out along the medial wall of the lateral femoral condyle. There are two bundles of the ACL—the anteromedial and the posterolateral, named according to where the bundles insert into the tibial plateau. The ACL attaches in front of the intercondyloid eminence of the tibia, being blended with the anterior horn of the lateral meniscus. These attachments allow it to resist anterior translation of the tibia, in relation to the femur.

Anterior cruciate ligament injury is the most common knee ligament injury, especially in athletes. Lateral rotational movements in sports like these are what cause the ACL to strain or tear. Anterior cruciate ligament (ACL) injury or Posterior Cruciate ligament (PCL) is normally treated by reconstruction which nowadays is done or assisted by arthroscopy. The ACL is the most commonly injured ligament of the knee and can be damaged in sports injuries or accidental injuries.

The ACL injury is followed by instability and repeated episodes of giving way which can damage the menisci and result in osteoarthritis or degeneration of the knee if left untreated.

The results of repair have been consistently unsuccessful; hence the ligament is replaced by various autologous grafts like the patellar tendon, hamstrings—Semitendinosus and/or the Gracilis, central third quadriceps or allografts. Recently, the hamstrings are becoming increasingly popular as their harvest does not follow morbidity.

The ACL has two distinct anatomic and functional bundles namely the Anteromedial (AM) bundle and the Posterolateral bundle (PL) named on the basis of their location on the tibia. The AM is the primary restraint against anterior translation of the tibia in flexion and the PL bundle is the primary restraint in extension. The two bundles cross each other in flexion with the AM bundle being posterior to the AL bundle in flexion and moving superior to the PL bundle in extension. In addition the ACL also provides rotational stability.

For reconstruction, two techniques are in use; namely:

(1) Single bundle ACL Reconstruction: where the native ACL is replaced by a quadrupled hamstring graft which is attached to the femur and tibia through single tunnels made in the anatomic centre of the native ACL. This is, by far, the most commonly performed surgery and is simpler to perform than the double bundle technique.

(2) Double bundle ACL Reconstruction: since the native ACL has two different bundles namely the AM and the PL bundles which are taut in different flexion angles a single bundle does not restore the original anatomy of the ACL as well as a double bundle ACL reconstruction.

In this technique, two separate tunnels are drilled into the femur and the tibia in their anatomic centres and two separate grafts are used to recreate the two bundles; so also the two grafts are fixed separately with separate implants in the femur and tibia.

There are reports of residual instability and pivot shift following single bundle reconstruction with early degeneration of the joint. This can be prevented by double bundle reconstruction presumably though no long term studies or evidence is yet available.

In addition to this, the size of the native ACL varies considerably and the size of the graft may not match it in single bundle reconstruction if the native ACL has a very large footprint.

Various implants are in vogue for fixing the soft tissue graft at the femur but can be broadly divided into (1) Suspensory cortical fixation outside the tunnel e.g. the endobutton or the Transfix

(2) Aperture fixation e.g. with interference screw.

It is well settled that the suspensory fixation provides a very strong and secure femoral fixation.

On the tibial side the graft can be secured with interference screw or tied over a suture disc or over a suture post with screw and washer.

The reconstruction requires technically demanding steps like femoral and tibial drilling and in case of Endobutton CL fixation a stepped socket needs to be drilled in the femur without which fixation with endobutton is impossible. Problems like posterior tunnel wall blowout are common and can only be avoided with technical detail.

The PCL is an intracapsular ligament along with the anterior cruciate ligament (ACL) because it lies deep within the knee joint. They are both isolated from the fluid-filled synovial cavity, with the synovial membrane wrapped around them. The PCL gets its name by attaching to the posterior portion of the tibia.

The function of the PCL is to prevent the femur from sliding off the anterior edge of the tibia and to prevent the tibia from displacing posterior to the femur. Common causes of PCL injuries are direct blows to the flexed knee, such as the knee hitting the dashboard in a car accident or falling hard on the knee, both instances displacing the tibia posterior to the femur.

A torn anterior cruciate ligament cannot be “repaired”, and must instead be reconstructed with a tissue graft replacement.

For reconstruction, a hole is drilled through the femur and tibia. The graft forming the ligament is guided through the drill hole and attached in place on the external walls of the bones, typically by endobutton on the femur and sutures tied over a post on the tibia (suspensory fixation). Alternatively the grafts may be secured inside the tunnels at the apertures with bioabsorbable screws or metallic screws (aperture fixation) in order to complete the process of attachment.

Advances in arthroscopy has led to the design and availability of buttons which hold the graft and sit across the drilled hole in the form of an anchor. In the current form of surgery, a hole of a defined diameter is drilled through the medial side of the femur in a transverse direction. After reaching the midpoint of the femur, a narrower tunnel is drilled to complete the hole through to the lateral side of the bone. A button sits as an anchor on this lateral side, atop the cavity defined by the hole.

However, it has been observed that the anchor buttons available in the market work on the pre-condition that the hole is accurately drilled in accordance with specified parameters of dimensions.

Depending upon the numerous kinds of cases and bone structure and size, it becomes difficult for the surgeon to drill actual textbook holes, in spite of precision equipment. It has been observed that ‘blowouts’ may occur, rendering hole diameters larger that the length of the anchor buttons available to the surgeon.

Smith and Nephew® has provided an Endobutton® regular without suture loops and Endobutton CL® with prefabricated knotless polyester loop of sizes from 15 mm to 60 mm which is an anchor button which caters to holes up to 10 mm (4.5 mm) in size. In order to counteract the blowout condition, Smith and Nephew® developed an Extendobutton® which is a larger anchor button which works in conjunction with the Endobutton®; and can cater to blowouts from 6 to 10 mm.

The Endobutton® snugs into the Extendobutton®, albeit leaving some amount of gap/play between the two devices. This play/play poses problems as the gravity of anchorage that it provides is susceptible to motion, thereby providing a not-so-good fit of the graft.

There is a need for a reliable button (or anchorage device) which provides unconditional stability.

In cases of double tunnel reconstruction, the Double Bundle PCL Guides give versatility in creating appropriate socket placement using anatomical constants or directly visualizing the intended socket diameter with the guides. Two holes side by side form the double tunnel to receive the double bundle.

The grafts are passed through respective tunnels for securing.

In surgeries, extreme conditions of blow out cannot be ruled out. These extreme cases may be rare cases, but there is a need for a salvaging method, and hence a product that can help a surgeon salvage complex and large blowout cases, with ease in order to provide peace of mind to the surgeon as well as allow a working model for the patient.

### OBJECTS OF THE INVENTION

An object of the invention is to provide a reliable anchorage device for holding on to a graft.

Another object of the invention is to provide a reliable and stable anchorage device for holding on to a graft in the event of a blowout of a tunnel in the bone.

Another object of the invention is to provide cortical fixation with the bridge button which allows separation of the two bundles (of grafts).

Yet another object of the invention is to provide an anchorage device for ligament reconstruction, which anchorage device is capable to be used in large blowout cases of the bone.

Still another object of the invention is to provide an anchorage device for ligament reconstruction, which anchorage device is capable to be used in complex blowout cases of the bone.

Still another object of the invention is to provide an anchorage device for ligament reconstruction, which anchorage device is capable to be used in revision cases of the ligament reconstruction.

Another object of the invention is to provide salvaging options to a surgeon during ligament reconstruction irrespective of the size of the hole or in cases of blowout.

### SUMMARY OF THE INVENTION

For the purposes of this invention, a ‘button’ relates to an anchorage device adapted to provide anchor support to a ligament graft. Typically, the button sits across the outer surface of the cavity of a hole through which the graft is passed; the graft can be attached to the button with ethibond sutures, mersilene tape, closed loop polyester loop or the tightrope.

For the purposes of this invention, a “bridge principle” may be defined as the longest distance or the “bridge” provided in between the two central holes of the implant for the suture loop housing the graft or in between the outer borders of the two strands of the loop holding the graft in case of single tunnel single bundle ACL reconstruction (or in between the outer borders of the two strands of two different loops of ethibond suture, mersilene tape, polyester or any suitable strong material like fiber wire used to hold the graft in case of double bundle ACL reconstruction using the Combihole technique with the Bridge button DT.)

According to this invention, there is provided an anchorage device or a button formed by an elongate body or multiple elongate bodies of pre-defined dimensions. The button body has pre-defined dimensions so that the button is adapted to sit across a cavity in a bone. This button may be referred to as BRIDGE BUTTON SMART.

This bridge is of critical importance to effectively space the suture loop inside the blowout tunnel; e.g. in a 10 mm cortical blowout an 8 mm bridge with the suture loops can effectively fill the tunnel internally by the suture loops leaving no space for side to side movement of the implant and effects rigid, secure, reliable and reproducible femoral cortical fixation. This also eliminates the risk of dislodgement or loss of fixation completely.

For the button of this invention, the ‘bridge principle’ is adapted to be used, wherein the span of the bridge is typically supported by means of support at extreme ends i.e. without any support in the centre or across the span of the bridge. The “bridge” principle, which defines the incorporation of a bridge, basically relies on the distance or the “Bridge” provided in between two pre-defined holes of an implant or in between the two strands of a single loop or in between the two strands of two different loops of ethibond suture, mersilene tape, polyester or any suitable strong material.

The bridge principle may be defined as the method of stabilizing a cortical button like the Bridge button onto the lateral cortical hole of the femoral tunnel particularly the blowout hole by spacing or spanning the loops of a suture, (ethibond suture or any suitable strong material like fibre wire, closed CL loop or tightrope etc) used for draping the graft (by) a distance nearly equaling the tunnel diameter. This eliminates side to side movement of the button, centralizes the button, and provides equal and adequate cover of the button on either side of the tunnel.

Alternatively, the button may be stabilized on the cortex by incorporating a collar almost equaling the tunnel diameter onto the undersurface of the button to engage securely inside the tunnel. This eliminates side to side movement of the button, centralizes the button, and provides equal and adequate cover on either side of the tunnel.

Methods of Increasing the Bridge:

(1) In the first method, the Bridge is provided in the implant design by increasing the span distance between the central two holes (meant for housing the loop for draping the graft) by a distance nearly equaling the tunnel diameter. Here the bridge principle basically relies on the bridge or “span distance” between the loops housing the graft. This Bridge in the implant design is critical to eliminate side to side movement of the button. This results in proper placement or centralization of the button, and equal and adequate cover of the button on either side of the tunnel orifice. e.g. in a 10 mm cortical blowout an 8 mm bridge along with the suture loops can effectively fill the tunnel internally by the suture loops leaving no space for side to side movement. Although the “Bridge” (BR) is primarily a function of the implant design, the thickness of the suture loops also adds to the effective bridge. The only link between the endobutton or bridge button and the exit hole cortex interface is the suture loop or CL loop holding the graft. Therefore the only way to stabilize the button on the lateral femoral cortex and to reduce its side to side movement (SSM) is by spanning the suture bridge and making it equal to the tunnel diameter. This is the “the bridge principle”. This centralizes the button, imparts unprecedented stability, and eliminates the risk of dislodgement of the button back into the tunnel completely. The Bridge buttons are designed to be used with 3 or 4 suture loops of simple No. 5 Ethibond suture or mersilene tape. However they are also compatible for use with a continuous polyester loop technology like the Endobutton CL (Smith & Nephew) or with a self adjusting suture loop technology like the TightRope (Arthrex, Naples) or any other suitable strong material like fibrewire etc.

(2) In the second method, the Bridge in a regular 4 hole button design or in a multihole button is increased by passing the suture loop housing the graft through the peripheral 2 holes instead of the normal central 2 holes. This also means that in a 4 hole Bridge button or in a multihole button like the Bridge Button Ultimate, it is possible to choose the bridge as per the tunnel diameter by passing the suture loops housing the graft through either the adjacent or the distant holes. The variable Bridge for varying tunnel situations can thus be used with advantage as per requirement e.g. in revision scenario.

(3) The third way to provide the bridge is to incorporate the bridge in the implant design by giving it an undersurface collar of 8 mm which can engage inside the tunnel securely without relying on the suture loops. In this case the implant may have holes separated by 2 mm or lesser bridge. The new extended bridge button with inbuilt bridge can be used with the regular endobutton CL just as the Xtendobutton. but does not exhibit side to side movement. The under collar engages in the tunnel mouth, prevents side to side movement and offers secure locking and fixation in a 10 mm tunnel. The XTB Bridge button can also be made compatible with any button like the Tight rope Button (Arthrex, Naples) or any similar button by modifying the bed on the top of the button to allow snug sitting of the respective button.

According to this invention, there is provided a bridge button, for ligament reconstruction, said button being formed by an arcuate body of pre-defined dimensions such that said button being adapted to sit across a cavity in a bone and further adapted to operate between an operative open position and an operative closed position, said button comprises:
a first shaft, and a co-axial second shaft co-operating with said first shaft in a manner such that said second shaft is adapted to be angularly displaced about said first shaft at their substantially co-axially located pivoting point, said second shaft being located operatively atop said first shaft.

According to this invention, there is further provided a bridge button, for ligament reconstruction, said button being formed by an arcuate body of pre-defined dimensions so that said button being adapted to sit across a cavity in a bone and further adapted to operate between an operative open position and an operative closed position, said button comprises:
a first shaft, and a co-axial second shaft co-operating with said first shaft in a manner such that said second shaft is adapted to be angularly displaced about said first shaft at their substantially co-axially located pivoting point, said second shaft being located operatively atop said first shaft; and
span member, adapted to define a bridge portion, said span member is defined by lateral edges of sutures placed across adjacent inner circumferences of a pair of pre-determined holes used for passing suture loop for holding a graft.

Preferably, said span member is a solid member, located substantially at the centre of said arcuate body of said button, adapted to define a bridge portion.

Typically, said span member is defined in accordance with the cavity it is adapted to sit across.

Typically, said span member is defined in accordance with the holes chosen for passing suture loops.

Typically, said span member comprises a plurality of holes for various purposes apart other than passing sutures for holding the graft.

Typically, said span member includes a plurality of holes at pre-determined locations.

Typically, said bridge button comprises a plurality of holes at pre-determined locations, beyond said bridge, adapted to allow passing of sutures for holding said graft, sutures for handling said button, sutures for handling an insert and the like purposes such that said holes may be selected in various combinations.

Typically, said bridge button comprises at least two holes for passing flipping sutures.

Typically, said bridge button comprises at least one hole for passing pulling sutures.

Typically, said bridge button comprises at least one hole for passing suture loop(s) for holding graft(s).

Typically, said button comprises a mechanism for facilitating angular displacement, said mechanism being provided at said pivoting point.

Preferably, said button comprises a stub at said pivoting point, said stub being adapted for co-operating in order to facilitate angular displacement.

Typically, said first shaft comprises a notch of a pre-defined arc along with a step such that said second shaft moves only by an angular displacement of 90 degrees and then locks itself in a pre-defined position in order to obtain a cross shaped assembly of an open configuration of said bridge button.

Typically, each of said shafts comprises holes and transverse ridges, therebetween, in order to facilitate suture passage.

Preferably, each of said shafts comprises lateral channels, in a longitudinal axial disposition, in order to accommodate suture passage such that sutures may pass and reside in the channel without affecting or hampering said angular displacement

Preferably, said first shaft comprises a lateral channel, in a longitudinal axial disposition, on the operative upper surface of said first shaft in order to accommodate suture passage such that sutures may pass and reside in said channel without affecting or hampering said angular displacement

Preferably, said second shaft comprises a lateral channel, in a longitudinal axial disposition, on the operative lower surface of said second shaft in order to accommodate suture passage such that sutures may pass and reside in said channel without affecting or hampering said angular displacement.

### BRIEF DESCRIPTION OF THE ACCOMPANYING DRAWINGS

The invention will now be described in relation to the accompanying drawings, in which:

FIGS. 1a and 1b illustrate a schematic of the lateral condyle of the femur bone with AM tunnel and PL tunnel according to different surgical procedures;

FIG. 2 illustrates a schematic of the AM tunnel and PL tunnel in the tibia;

FIGS. 3a, 3b, and 3c illustrate a schematic of various AM portals and PL portals in the tibia;

FIGS. 4a, 4b, 4c, 4d, 4e illustrate various combinations of holes of the tunnel that are drilled for surgery; and

FIGS. 5a-5f illustrates yet another anchorage device or button for ligament reconstruction.

### DETAILED DESCRIPTION OF THE ACCOMPANYING DRAWINGS

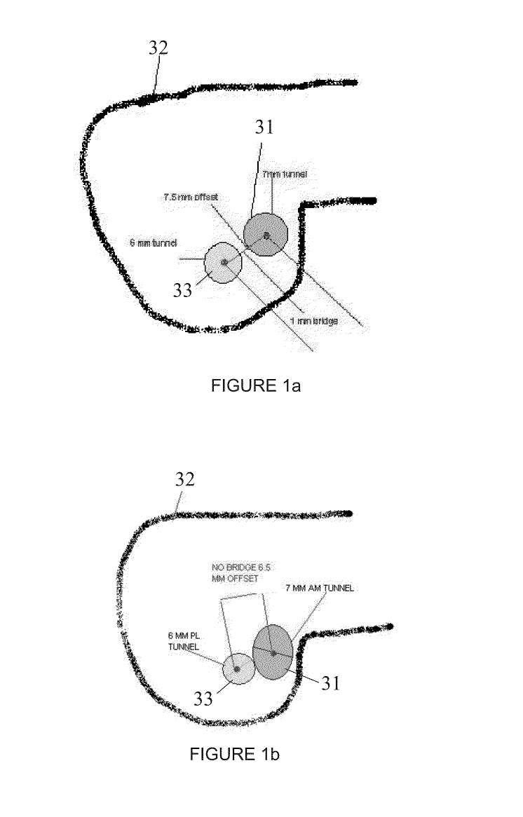

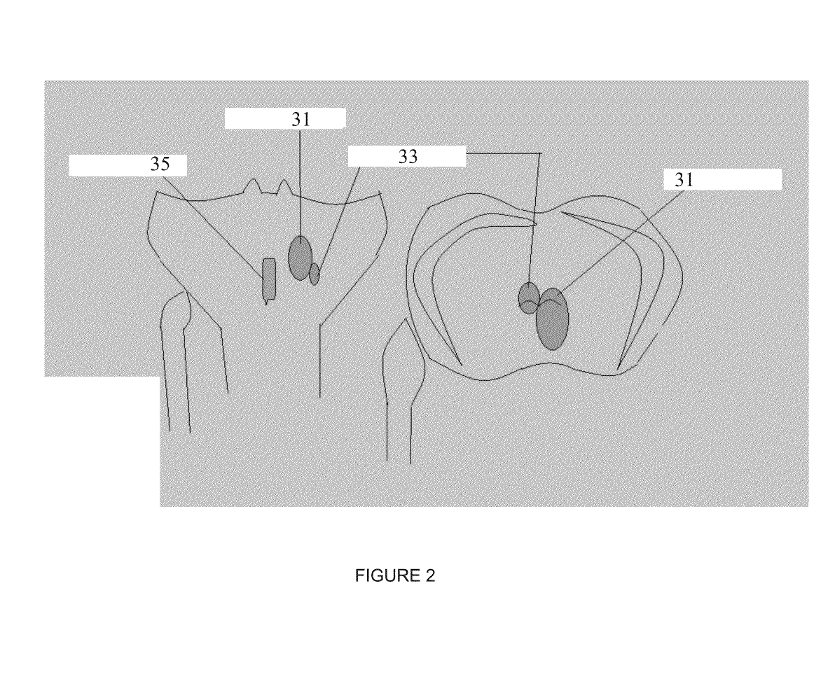

FIGS. 1a and 1b illustrate a schematic of the lateral condyle (32) of a femur bone with AM tunnel (31) and PL tunnel (33) according to different surgical procedures. FIG. 2 illustrates a schematic of the AM tunnel (31) and PL tunnel (33) in the tibia. Reference numeral 35 refers to tuberal tuberosity.

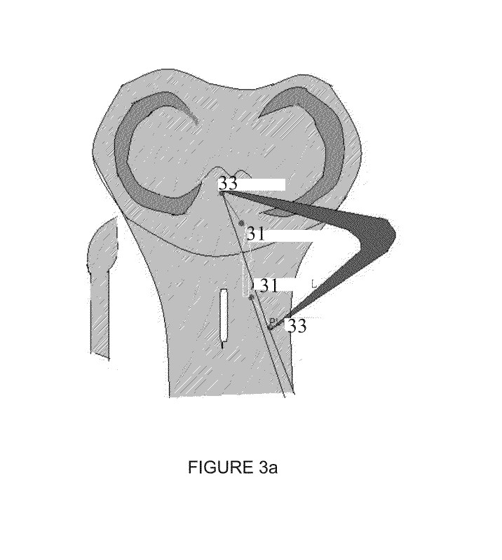

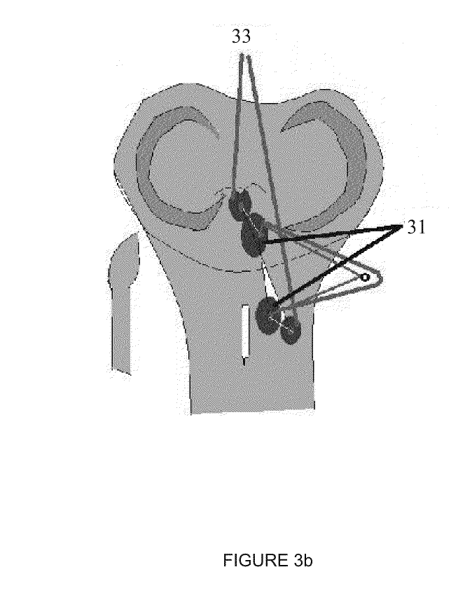

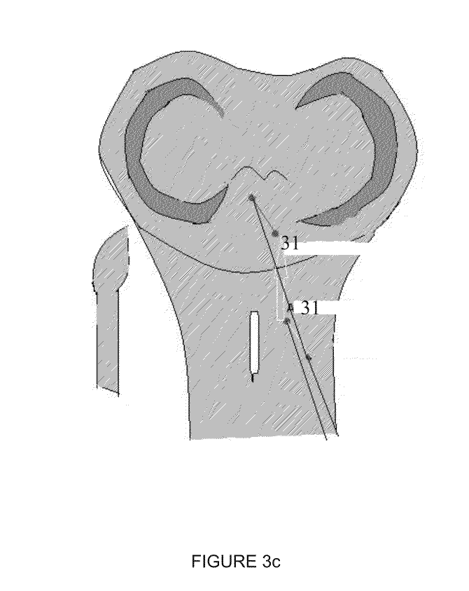

FIGS. 3a, 3b, and 3c illustrate a schematic of various AM tunnel and PL tunnel in the tibia.

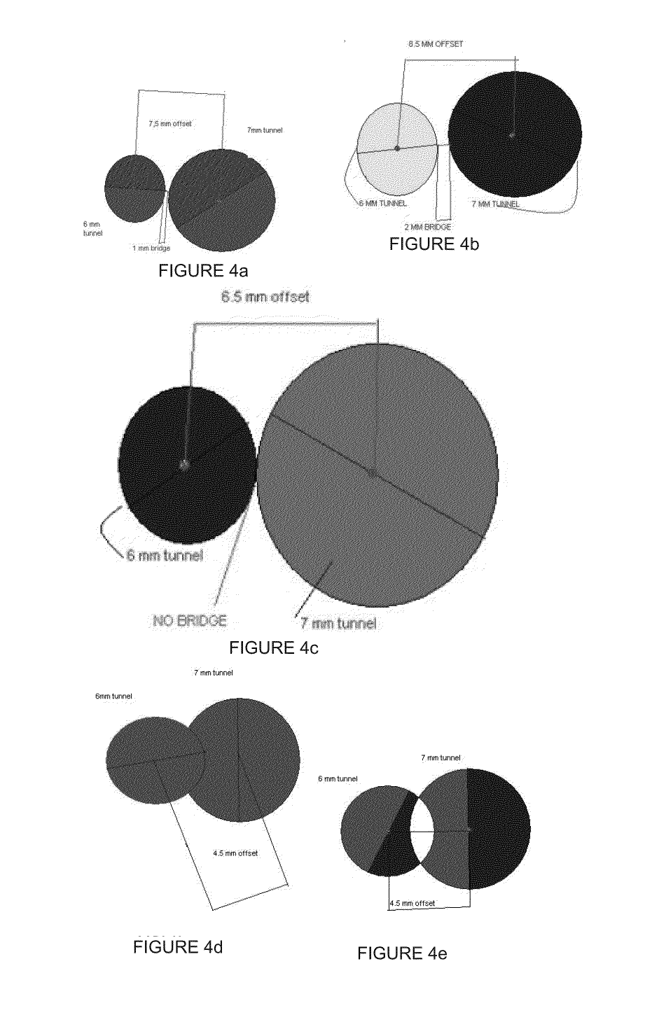

FIGS. 4a, 4b, 4c, 4d, 4e illustrate various combinations of holes of the tunnel that are drilled for surgery.

Although the “Bridge” is primarily a function of the implant design, the thickness of the suture loops adds to the effective bridge. It is possible to increase the Bridge in a 4-hole design or a multi-hole button by passing the sutures through the peripheral 2 holes instead of the routine central 2 holes.

This means that in a multi-hole button (like the 4 hole BB or Bridge button Ultimate) it is possible to choose the bridge as per the tunnel diameter by passing the suture loops housing the graft through either the adjacent or the distant holes. The only link between the endobutton or bridge button and the exit hole cortex interface is the suture loop or CL loop holding the graft. Therefore, the only way to stabilize the button on the lateral femoral cortex and to reduce its side to side movement is by spanning the suture bridge using the bridge principle. This imparts unprecedented stability and eliminates the risk of dislodgement of the button back into the tunnel completely. This bridge in the implant design is of critical importance to effectively space the suture loop inside the blowout tunnel; e.g. in a 10 mm cortical blowout an 8 mm bridge along with the suture loops can effectively fill the tunnel internally by the suture loops leaving no space for side to side movement of the implant and effects rigid, secure, reliable and reproducible femoral cortical fixation. The Bridge buttons are designed to be used with 3 or 4 suture loops of simple No. 5 Ethibond suture or mersilene tape with or without ethibond loops. However, they may be compatible for use with a continuous polyester loop technology like the Endobutton CL (SMITH & NEPHEW) or with a self locking suture loop technology like the TightRope (ARTHREX, NAPLES) or any other suitable strong material like fibrewire or the like.

For the purposes of this specification, ‘cover’ may be defined as the amount of bone covered by the implant after fixation and depends on the bridge provided in the implant design and the length of the implant. (In practice, the thickness of the suture loops adds to this bridge in the implant design.)

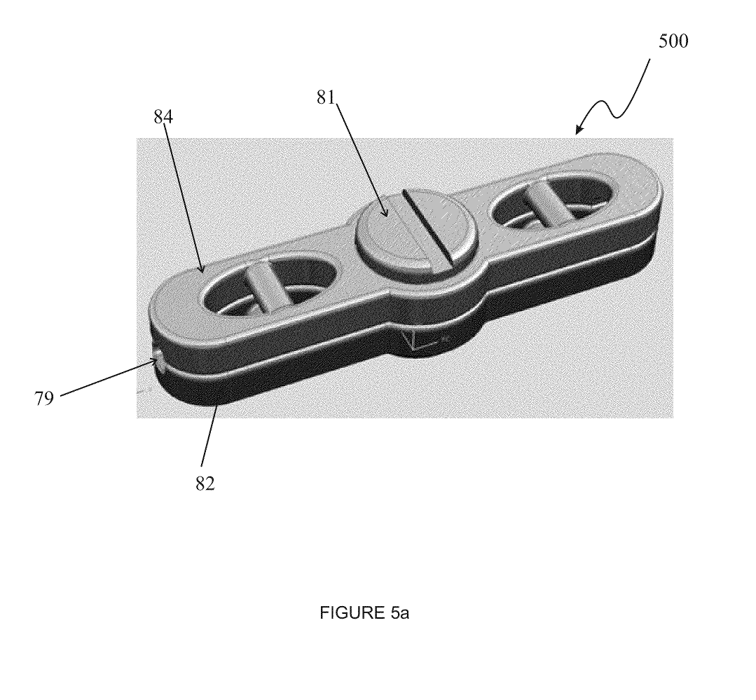

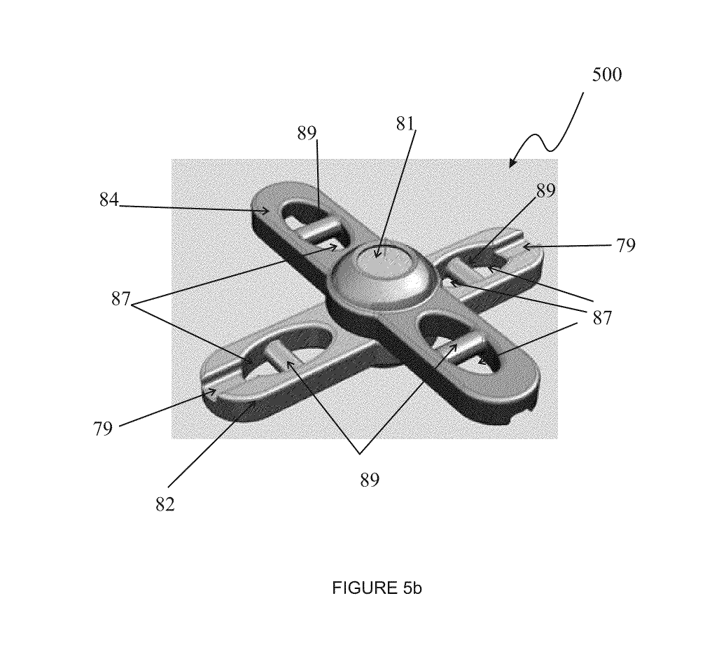

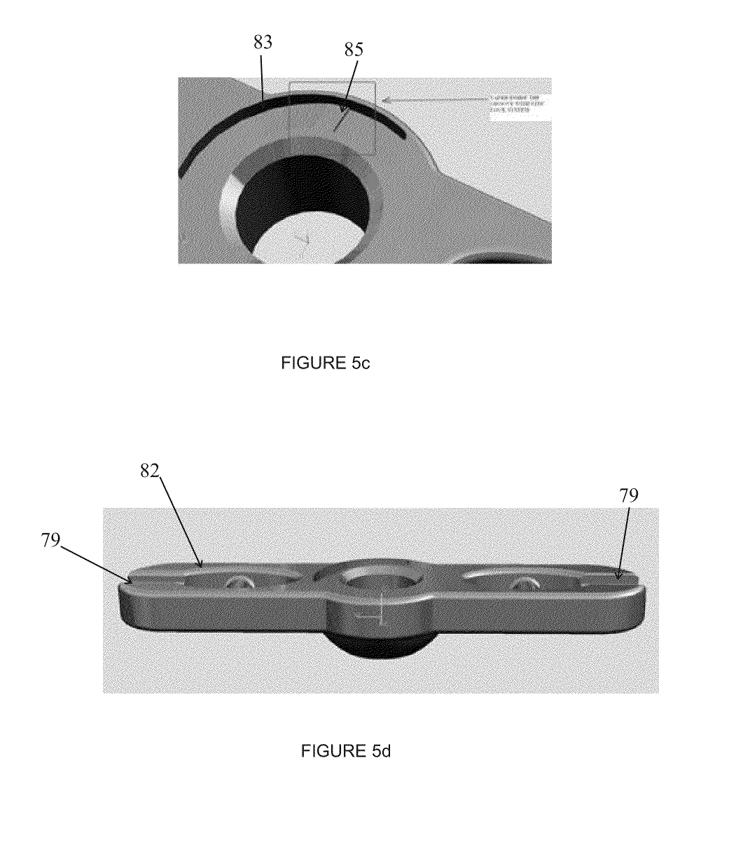

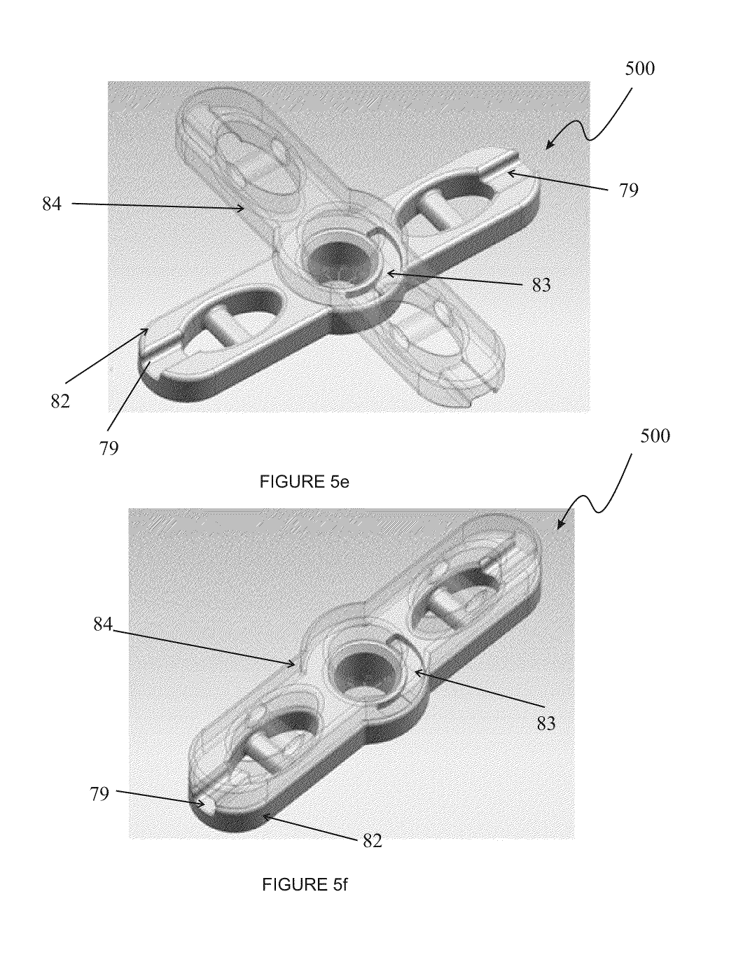

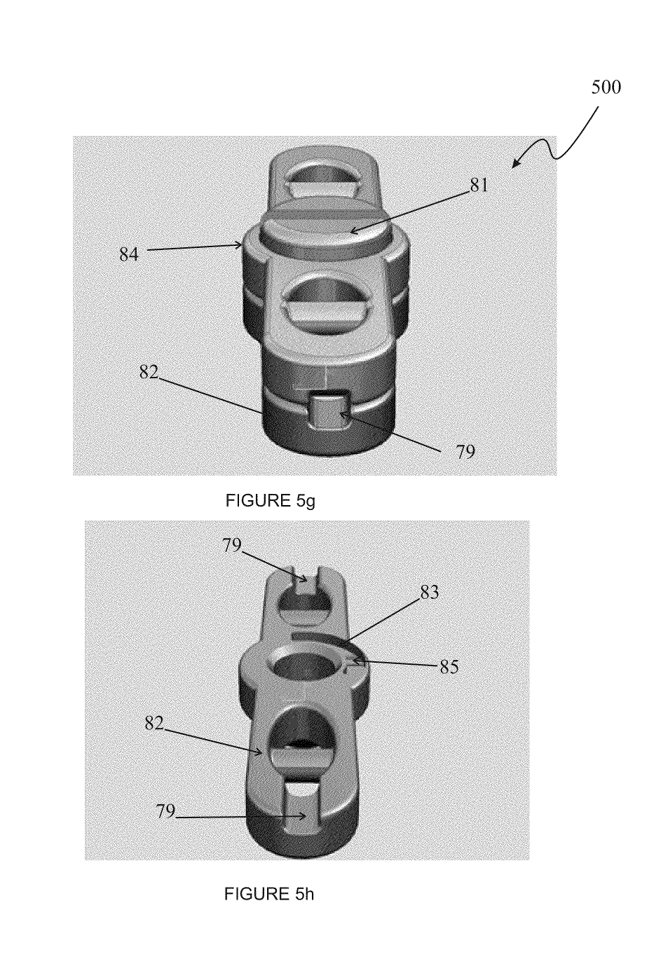

According to this invention, there is provided still an additional anchorage device or button for ligament reconstruction. (BRIDGE BUTTON SMART). Reference numeral 500 refers to such anchorage device or button for ligament reconstruction as seen in FIGS. 5a-5f of the accompanying drawings.

FIG. 5a refers to a closed configuration of this button, while FIG. 5b refers to an open configuration of this button. FIGS. 5c, 5e, 5f, 5h illustrate the mechanism which leads to opening and locking of the button from the closed configuration to the open configuration.

In accordance with an embodiment of this invention, there is provided a first shaft (82) and a second shaft (84) co-operating with said first shaft in a manner such that the second shaft is adapted to be angularly displaced about the first shaft at their substantially centrally located pivoting point (81). A mechanism for angular displacement is provided at the pivoting point. The second shaft lays operatively atop the first shaft and is able to angularly displace in a slideable manner over the first shaft. There is a substantially round part in the centre of each of the shafts which form the mechanism (81) for co-operating in order to facilitate angular displacement. The first shaft includes a notch (83) of pre-defined arc along with a step (85) such that the second shaft moves only by an angular displacement of 90 degrees and then locks itself in the position. Each shaft includes holes (87) and transverse ridges (89) in order to facilitate suture passage.

There are further provided lateral channels (79) in an axial disposition in the first shaft such that sutures may pass and reside in the channel without affecting or hampering the angular displacement.2

It is a strong coupling of two buttons which allows the buttons to rotate and lock at 90 degrees once flipped creating a cross design which can be used in complex large blowouts especially in DBACLR.

Ideally, the bridge element, in every button or anchorage device, sits across the cavity with the lateral exterior-most holes placed outside the cavity and resting upon the solid portion of the bone.

In a preferred embodiment and method, the bridge is marginally lesser than the cavity. The margin which is the differential incorporates the diameter of the sutures on either end. Thus the sutures pass through the inner edge of the holes and are plugged in between said inner edge of the hole and the wall of the tunnel. This provides a secure fit of the button onto the cavity.

In a preferred embodiment, the holes are placed on either side of said solid bridge element thereby providing the bridge formed by the solid element for providing a salvaging component in lateral femoral cortex blowout conditions in ACL or PCL reconstruction.

The confluence of two tunnels is labelled “combihole” because it resembles the shape of the screw holes in a locking plate with combihole for locking or dynamic hole options.

This implant or the Bridge Button provides a very simplified technique of the complex double bundle ACL reconstruction which many single bundle surgeons may find easy to perform and also provides a backup salvage implant in case of complications like tunnel communication or blowout.

The versatility of the implant makes it conducive to be included in the inventory of any Orthopaedic surgeon treating ACL or PCL tear.

The Bridge button is not only an alternative implant but an independent salvage implant catering to blowouts from 6 mm to 17 mm.

Any implant claiming to salvage a blowout tunnel must follow the bridge principle and if it does not do so it is doomed for failure as assessed by the “challenge test”.

The AM bundle is posterior on the femoral condyle to the PL bundle in knee flexion. With the Bridge button technique it is possible to rotate the bundles to orient them anatomically to their respective positions which may not be possible with any other technique or implants; hence it is the only and unique method to restore the two bundle anatomy without the difficulties in conventional double bundle ACL reconstruction surgery.

With the advent of anatomic ACL reconstruction the femoral tunnel tends to be short at times critically short that is less than 25 mm short where Endobutton CL® with smallest loop of 15 mm is impossible to use. The free hand technique with manual loop knotting or the Arthrex TightRope® technology is the only way to address tunnels smaller than 25 mm apart from the Endobutton Direct® from Smith & Nephew® which cannot provide a bridge to separate the two bundles and can be used only for single bundle ACL reconstruction.

The smaller the tunnel and hence the loop the greater the difficulty in maneuvering the Bridge button out the femoral tunnel. The TightRope® technology can permit a longer loop for locking of the Bridge button and allow individual passage of the two bundles with more ease.

Alternatively, the tendons can be separated with a Bioscrew/combicage to allow separation of the two bundles or a RCI Screw.

The PCL reconstruction is done on the same principles using the bridge button and some special instruments.

The versatility of the implant makes it an absolute must in the inventory of any Orthopedic surgeon treating ACL or PCL TEARS.

The Bridge button is not only an alternative implant but also a salvage implant catering to blowouts from 6 mm to 17 mm.

The technical advancement lies in the fact that this fixture (bridge button) is a dual assembly which provides a firm placement across a cavity of the bone without any scope for dislodgement or failure. Further, it can be used in minimally invasive procedures and in complex or salvaging procedures with a relatively greater degree of stability.

Generally, in case of lateral cortex blowout a suture disc or post can be used to fix the graft on the lateral femur but this requires additional incision on the lateral thigh, is time consuming and adds to the pain and scar. With the Bridge button no lateral incision is required and the button can be passed through the tibial and femoral tunnels itself

One implant does it all from single tunnel single bundle to double tunnel double bundle surgery in addition to providing salvage backup in cases of cortical blowouts

While considerable emphasis has been placed herein on the particular features of this invention, it will be appreciated that various modifications can be made, and that many changes can be made in the preferred embodiments without departing from the principles of the invention. These and other modifications in the nature of the invention or the preferred embodiments will be apparent to those skilled in the art from the disclosure herein, whereby it is to be distinctly understood that the foregoing descriptive matter is to be interpreted merely as illustrative of the invention and not as a limitation.

1. A bridge button, for ligament reconstruction, said button being formed by an arcuate body of pre-defined dimensions such that said button being adapted to sit across a cavity in a bone and further adapted to operate between an operative open position and an operative closed position, said button comprising:
a first shaft, and a co-axial second shaft co-operating with said first shaft in a manner such that said second shaft is adapted to be angularly displaced about said first shaft at their substantially co-axially located pivoting point, said second shaft being located operatively atop said first shaft.

2. A bridge button, for ligament reconstruction, as claimed in claim 1 wherein, said button comprising a mechanism for facilitating angular displacement, said mechanism being provided at said pivoting point.

3. A bridge button, for ligament reconstruction, as claimed in claim 1 wherein, said button comprising a stub at said pivoting point, said stub being adapted for co-operating in order to facilitate angular displacement.

4. A bridge button, for ligament reconstruction, as claimed in claim 1 wherein, said first shaft comprising a notch of a pre-defined arc along with a step such that said second shaft moves only by an angular displacement of 90 degrees and then locks itself in a pre-defined position in order to obtain a cross shaped assembly of an open configuration of said bridge button.

5. A bridge button, for ligament reconstruction, as claimed in claim 1 wherein, each of said shafts comprising holes and transverse ridges, therebetween, in order to facilitate suture passage.

6. A bridge button, for ligament reconstruction, as claimed in claim 1 wherein, each of said shafts comprising lateral channels, in a longitudinal axial disposition, in order to accommodate suture passage such that sutures may pass and reside in the channel without affecting or hampering said angular displacement

7. A bridge button, for ligament reconstruction, as claimed in claim 1 wherein, said first shaft comprising a lateral channel, in a longitudinal axial disposition, on the operative upper surface of said first shaft in order to accommodate suture passage such that sutures may pass and reside in said channel without affecting or hampering said angular displacement

8. A bridge button, for ligament reconstruction, as claimed in claim 1 wherein, said second shaft comprising a lateral channel, in a longitudinal axial disposition, on the operative lower surface of said second shaft in order to accommodate suture passage such that sutures may pass and reside in said channel without affecting or hampering said angular displacement

9. A bridge button, for ligament reconstruction, said button being formed by an arcuate body of pre-defined dimensions so that said button being adapted to sit across a cavity in a bone and further adapted to operate between an operative open position and an operative closed position, said button comprising:
a first shaft, and a co-axial second shaft co-operating with said first shaft in a manner such that said second shaft is adapted to be angularly displaced about said first shaft at their substantially co-axially located pivoting point, said second shaft being located operatively atop said first shaft; and
span member, adapted to define a bridge portion, said span member being defined by lateral edges of sutures placed across adjacent inner circumferences of a pair of pre-determined holes used for passing suture loop for holding a graft.

10. A bridge button as claimed in claim 9 wherein, said span member being a solid member, located substantially at the centre of said arcuate body of said button, adapted to define a bridge portion.

11. A bridge button as claimed in claim 9 wherein, said span member being defined in accordance with the cavity it is adapted to sit across.

12. A bridge button as claimed in claim 9 wherein, said span member being defined in accordance with the holes chosen for passing suture loops.

13. A bridge button as claimed in claim 9 wherein, said span member comprising a plurality of holes for various purposes apart other than passing sutures for holding the graft.

14. A bridge button as claimed in claim 9 wherein, said span member includes a plurality of holes at pre-determined locations.

15. A bridge button as claimed in claim 9 wherein, said bridge button comprising a plurality of holes at pre-determined locations, beyond said bridge, adapted to allow passing of sutures for holding said graft, sutures for handling said button, sutures for handling an insert and the like purposes such that said holes may be selected in various combinations.

16. A bridge button as claimed in claim 9 wherein, said bridge button comprising at least two holes for passing flipping sutures.

17. A bridge button as claimed in claim 9 wherein, said bridge button comprising at least one hole for passing pulling sutures.

18. A bridge button as claimed in claim 9 wherein, said bridge button comprising at least one hole for passing suture loop(s) for holding graft(s).

19. A bridge button, for ligament reconstruction, as claimed in claim 9 wherein, said button comprising a mechanism for facilitating angular displacement, said mechanism being provided at said pivoting point.

20. A bridge button, for ligament reconstruction, as claimed in claim 9 wherein, said button comprising a stub at said pivoting point, said stub being adapted for co-operating in order to facilitate angular displacement.

21. A bridge button, for ligament reconstruction, as claimed in claim 9 wherein, said first shaft comprising a notch of a pre-defined arc along with a step such that said second shaft moves only by an angular displacement of 90 degrees and then locks itself in a pre-defined position in order to obtain a cross shaped assembly of an open configuration of said bridge button.

22. A bridge button, for ligament reconstruction, as claimed in claim 9 wherein, each of said shafts comprising holes and transverse ridges, therebetween, in order to facilitate suture passage.

23. A bridge button, for ligament reconstruction, as claimed in claim 9 wherein, each of said shafts comprising lateral channels, in a longitudinal axial disposition, in order to accommodate suture passage such that sutures may pass and reside in the channel without affecting or hampering said angular displacement

24. A bridge button, for ligament reconstruction, as claimed in claim 9 wherein, said first shaft comprising a lateral channel, in a longitudinal axial disposition, on the operative upper surface of said first shaft in order to accommodate suture passage such that sutures may pass and reside in said channel without affecting or hampering said angular displacement

25. A bridge button, for ligament reconstruction, as claimed in claim 9 wherein, said second shaft comprising a lateral channel, in a longitudinal axial disposition, on the operative lower surface of said second shaft in order to accommodate suture passage such that sutures may pass and reside in said channel without affecting or hampering said angular displacement
